(19) United States
(12) Patent Application Publication
(10) Pub. No.: US 2026/0283798 A1
(43) Pub. Date: Sep. 24, 2026

(54) DEVICE FOR ENABLING TRANS-CATHETER IMPLANTATION

(71) Applicant: CORQUEST MEDTECH, Ottignies-Louvain-la-Neuve (BE)

(72) Inventors: Arnaud VAN SCHEVENSTEEN, Ixelles (BE); Christophe MOUREAUX, Besancon (FR); Olivier DECUYPERE, Bruxelles (BE); Sébastien DAVID, Besancon (FR); Xavier BOLLEN, Namur (BE); Ludovic MELLY, Malonne (BE); Didier DE CANNIÈRE, Rhode-Saint-Genèse (BE)

(21) Appl. No.: 19/168,160

(22) PCT Filed: Mar. 29, 2024
(86) PCT No.: PCT/EP2024/058764

§ 371 Date: Sep. 23, 2025

(30) Foreign Application Priority Data
Mar. 31, 2023 (BE) BE2023/5250

(51) Int. Cl. A61F 2/24

(52) U.S. Cl. CPC A61F 2/2454; A61F 2210/0014; A61F 2220/0016

(57) ABSTRACT

A device comprising a body having a central part from which an anterior part and a posterior part extend. The posterior part comprises at least 2 elements and an end which is distal with respect to the central part. The anterior part comprises at least 1 longitudinal element having a proximal end connected to the central part and an end which is distal with respect to the central part. Each of the distal ends of the at least 1 or 2 longitudinal elements is in a raised position with respect to the central part. The device is useful for enabling trans-catheter implantation. A kit having the device and a medical device, e.g., a catheter, is also provided. Moreover, a method for transcatheter implantation of a medical device, e.g., a catheter, is provided.

The present invention relates to a medical device arranged to allow transcatheter implantation to treat mitral regurgitation.

Generally, known devices may take the form of a ring that is inserted by open heart surgery, which has several disadvantages, particularly in terms of mortality risk.

Thus, other solutions have focused on transcatheter implantation, which requires the ability to move the medical device in a confined space, such as a catheter, and then deploy it during the implantation procedure. For example, such a device may take the form of an atrioventricular valve repair ring, on which a barrier is assembled that enables blood flow after delivery of the device. Transcatheter clips also exist.

Generally, known devices take the form of a kit or devices that may include several elements that need to be assembled in order to be functional. These known devices still present difficulties in terms of positioning and implantation. They are often recommended for a very specific patient population and therefore cannot be used to treat a large number of patients or a wide range of mitral pathologies.

WO 2020/154797 describes a device for treating mitral regurgitation that features a ring and cords.

WO 2020 167 672 relates to a system for enabling the percutaneous implantation of a medical device within a heart valve.

For these reasons, there is a real need to provide a medical device to treat mitral regurgitation that is simple in terms of its proposed structure and effective in terms of its functionality so as to effectively treat mitral regurgitation. The handling and delivery of the device also needs to be improved.

To solve this problem, the present invention provides a device arranged to enable transcatheter implantation and to treat mitral regurgitation, comprising a body (1) having a central portion (2) from which an anterior portion (3) and a posterior portion (4) extend, said device having a folded position arranged to be held in a medical device in the form of a flexible tube, preferably catheter, and a deployed position arranged to treat mitral regurgitation, and, wherein, in the deployed position:
said posterior portion (4) being arranged to be placed above the posterior mitral leaflet and arranged to be fixed on the posterior side of the mitral valve and comprising at least 2, preferably at least 3, longitudinal elements (5), each of which has a proximal end (5a) connected to said central portion (2) and a distal end (5b) relative to said central portion (2) so that said at least 2 longitudinal elements (5) each extend away from said central portion (2),
said anterior portion (3) being arranged to be placed above the anterior mitral leaflet and arranged to be fixed on the anterior side of the mitral valve and comprising at least 1 longitudinal element (6) (preferably at least 2 longitudinal elements) which has a proximal end (6a) connected to said central portion (2) and a distal end (6b) relative to said central portion (2), so that said at least 1 longitudinal element (6) extends away from said central portion (2) and also said at least 2 longitudinal elements (5), and wherein
the distal end (6b) of said at least 1 longitudinal element (6) and/or each of said distal ends (5b) of said at least 2 longitudinal elements (5) being in a raised position relative to said central portion (2) which is located in a longitudinal axis (Al) relative to the length (L) of said body (1).

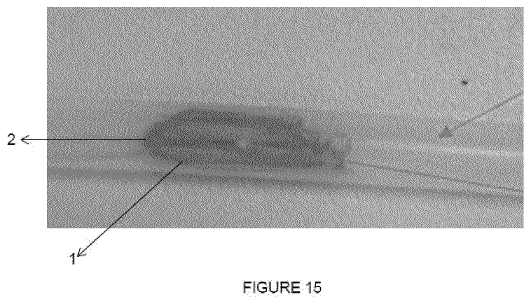

Thus, the device according to the invention has a first folded position in which it can be securely held, for example, in a catheter (see FIG. 15). Once the device is in the deployed position, it has a relatively simple and efficient structure in terms of functionality.

Preferably, said at least two longitudinal elements (5), preferably said at least three longitudinal elements (5), more preferably said at least four longitudinal elements (5), even more preferably said at least five longitudinal elements, advantageously said at least six longitudinal elements, of the posterior portion (4) are separated from one another so as to form separate elements.

Advantageously, when there are at least two, preferably at least three, more preferably at least four longitudinal elements (6) of the anterior portion (3), they are separated from one another so as to form separate elements. The posterior portion (4) has a number of longitudinal elements (5) ranging from 2 to 20, preferably from 2 to 18, more preferably from 2 to 16, and even more preferably from 2 to 12. These are advantageously separated from one another so as to form separate elements.

The anterior portion (3) has a number of longitudinal elements (6) ranging from 1 to 18, preferably from 1 to 15, and even more preferably from 1 to 12. These are advantageously separated from one another so as to form separate elements.

Advantageously, the posterior portion (4) has (at least) 6 longitudinal elements (5) and the anterior portion (3) has (at least) 2 longitudinal elements (6). These are advantageously separated from one another so as to form separate elements.

These embodiments with regard to the number of longitudinal elements (5, 6) of the anterior (3) and posterior (4) portions can be applied to all the other preferred embodiments listed below.

This makes it possible to provide a device that takes up a certain amount of space, which makes it easier to handle.

In a particularly preferred manner, said at least one longitudinal element (6) of the anterior portion (3) and said at least two longitudinal elements (5) of the posterior portion (4) each have a hollow shape (7) arranged to allow blood flow, in the deployed position. This means that blood flow can take place through each of the aforementioned longitudinal elements.

Even more preferably, said at least one longitudinal element (6) has a hollow internal portion (13b) and a solid external portion (13a), the hollow internal portion (13b) defining a surface area that is larger than the surface area defined by the solid external portion (13a). The external portion (13a) takes the form of a segment extending around the circumference of said longitudinal element.

Advantageously, said at least two longitudinal elements (5) each have a hollow internal portion (12b) and a solid external portion (12a), the hollow internal portion (12b) defining a surface area that is larger than the surface area defined by the solid external portion (12a). The external portion (12a) is preferably in the form of a segment extending around the circumference of said longitudinal element.

Advantageously, the posterior portion (4) comprises a greater number of longitudinal elements (5) than the number of longitudinal elements (6) present in the anterior portion (3). This achieves a superior clinical effectiveness than that of known devices. This embodiment provides better coverage of the posterior region of the valve, which may have more or less numerous and more or less marked indentations, during delivery, which allows for better management of possible prolapses of the posterior leaflet of the valve.

In a particularly advantageous embodiment, in the deployed position, said distal ends (6b) of said at least one longitudinal element (6) of the anterior portion (3) are in a position raised by a height of between 0.5 and 15 mm, preferably between 0.5 and 10 mm, more preferably between 3 and 10 mm, relative to said central portion (2) which is located in a longitudinal axis (A1) relative to the length (L) of said body (1).

Thus, when the device is used to treat mitral regurgitation, the pressure applied to the anterior side of the mitral valve is such that the device can be effectively held in position over time.

Preferably, in the deployed position, said distal ends (5b) of said at least 2 longitudinal elements (5) of the posterior portion (4) being in a position raised by a height of between 0.5 and 15 mm, preferably between 0.5 and 10 mm, more preferably between 3 and 10 mm, relative to said central portion (2) which is located in a longitudinal axis (Al) relative to the length (L) of said body. Thus, when the device is used to treat mitral regurgitation, the pressure applied to the posterior side of the mitral valve is such that the device can be effectively held in position over time.

Advantageously, when the device according to the invention is in the deployed position on the mitral valve, all the ends (5b, 6b) of the respective longitudinal elements (5, 6) rest (in part) on the mitral annulus, while remaining free from one another. This means that there are no connecting means connecting said free ends of the longitudinal elements to each other when the device is in the deployed position on the mitral valve.

Advantageously, said at least one longitudinal element (6) of the anterior portion (3) and said at least two longitudinal elements (5) of the posterior portion (4) have a curved shape.

Advantageously, said central portion (2) is provided with an opening (11) arranged to receive a delivery means to bring the device above the mitral leaflets. This allows for easy positioning, confirmation of the size of the implant before anchoring, and immediate clinical functionality.

In this way, it is possible, for example, to insert a guide, such as a wire, designed to orient the device in the space, preferably at the level of the native mitral annulus.

Even more advantageously, said body (2) is molded in a single piece, preferably after laser or water jet cutting, in the form of a sheet of a material, advantageously with shape memory or superelastic, intended for medical use. The shape can thus be cut from a sheet of metal (e.g. nitinol) using a laser or water jet and then placed in a high-temperature thermoforming mold to then undergo rapid quenching.

The body is advantageously made of a biocompatible material selected from the group comprising alloys, nitinol, titanium, stainless steel, polymer material, and mixtures thereof.

According to a particularly preferred embodiment, the thickness of the body (1) is between 0.01 and 1 mm, preferably between 0.1 and 0.5 mm, more preferably between 0.2 and 0.4 mm, even more preferably between 0.2 and 0.3 mm, advantageously between 0.2 and 0.35 mm. The thickness ensures the implant remains strong while being able to follow and withstand movements and forces when it is placed. The thinness minimizes the presence of foreign bodies while ensuring that the device is effective. Furthermore, it is so flexible that it can also be folded into a catheter without losing effectiveness when in the deployed position.

In an advantageous embodiment, said at least one longitudinal element (6) of the anterior portion (3) and said at least two longitudinal elements (5) of the posterior portion (4) have the shape of lobes, petals, or branches.

Preferably, said longitudinal elements (5) of the posterior portion (4) of said device, in the deployed position, extend so that the distal ends (5b) form an arc of a circle.

Advantageously, the distal end (6b) of said at least one longitudinal element (6) of the anterior portion (3) and each distal end (5b) of said at least two longitudinal elements (5) of the posterior portion (4) are provided with a fixing means or a receiving means (8, 9) arranged to receive an attachment means (10).

Preferably, the device according to the invention also comprises at least one attachment means (10) for fixing it to a native mitral annulus.

According to a preferred embodiment, the fixing means or the attachment means (10) of said at least one longitudinal element (6) of the anterior portion (3) is/are arranged to be placed above the anterior mitral leaflet or annulus and fixed to the anterior side of the mitral valve.

According to an even more preferred embodiment, the fixing means or the attachment means (10) of the at least two longitudinal elements (5) of the posterior portion (4) is/are arranged to be placed above the posterior mitral leaflet or annulus and fixed to the posterior side of the mitral valve.

Advantageously, the fixing means or attachment means (8, 9) of their respective longitudinal elements (5, 6) are independent of each other, preferably not being connected to each other by any means.

According to an even more advantageous embodiment, said attachment means (10), arranged to be connected to said receiving means (8), has a distal attachment end (10a) arranged to be fixed to the native mitral annulus and a proximal end (10b), optionally provided with a connection means (10b) arranged to hold said attachment means (10) in said receiving means (8).

Preferably, said attachment means (10) has a first position arranged to allow it to be introduced into said receiving means (8) and a second position arranged to hold it in said receiving means (8), when it is fixed on the native mitral annulus in a fixed and stable manner over time.

According to a particular embodiment of the device according to the invention, in the folded position, it is arranged to be held in a medical device in the form of a flexible tube, preferably a catheter, which has an external diameter of between 3 and 15 mm, preferably between 4 and 12 mm, more preferably between 6 and 10 mm, and even more preferably between 6 and 8 mm.

The present invention also relates to a kit which comprises a device according to the invention and a medical device in the form of a flexible tube, preferably a catheter, having an external diameter of between 3 and 15 mm, preferably between 4 and 12 mm, more preferably between 6 and 10 mm, even more preferably between 6 and 8 mm, in which the device according to the invention is held in the folded position in said medical device.

Other embodiments of the kit will be detailed below.

The present invention also relates to a method of transcatheter implantation using the device according to the invention for treating mitral regurgitation, comprising the following steps:
Providing the device which comprises a body having a central portion (2) from which extends an anterior portion (3) and a posterior portion (4), said body (1) being in a folded position in which said body is held in a first medical device in the form of a flexible tube, preferably a catheter,
Bringing the device in the folded position into the left atrium of the heart by the vascular route, preferably via the venous, transfemoral or transseptal route,
Deploying the device so that it adopts a deployed position and orienting it above the mitral valve with:
   said posterior portion (4) of said body being placed above the posterior mitral leaflet and fixed on the posterior side of the mitral valve, said posterior portion comprising at least 2 longitudinal elements (5) each of which has a proximal end (5a) connected to said central portion (2) and a distal end (5b) relative to said central portion (2) so that said at least 2 longitudinal elements (5) each extend away from said central portion (2), and with:
   said anterior portion (3) being arranged to be placed above the anterior mitral leaflet and arranged to be fixed on the anterior side of the mitral valve and comprising at least 1 longitudinal element (6) (preferably at least 2 longitudinal elements) which has a proximal end (6a) connected to said central portion (2) and a distal end (6b) relative to said central portion (2) so that said at least 1 longitudinal element (6) extends away from said central portion (2) and also said at least 2 longitudinal elements (5), and
Stable and fixed delivery of the device above the mitral valve leaflets and removal of said medical device in the form of a flexible tube, preferably a catheter.

Advantageously, the device delivery step is performed using a second medical device, in the form of a flexible tube, such as a delivery catheter, acting as a delivery device.

During the removal step, the first and second medical devices are removed from the body. Preferably, the second (delivery) medical device is removed before the first medical device.

Preferably, said at least two longitudinal elements (5), preferably said at least three longitudinal elements (5), more preferably said at least four longitudinal elements (5), even more preferably said at least five longitudinal elements, advantageously said at least six longitudinal elements, of the posterior portion (4) are separated from one another so as to form separate elements.

Advantageously, when there are at least two, preferably at least three, more preferably at least four longitudinal elements (6) of the anterior portion (3), they are separated from one another so as to form separate elements.

The posterior portion (4) has a number of longitudinal elements (5) ranging from 2 to 20, preferably from 2 to 18, more preferably from 2 to 16, and even more preferably from 2 to 12. These are advantageously separated from one another so as to form separate elements. The anterior portion (3) has a number of longitudinal elements (6) ranging from 1 to 18, preferably from 1 to 15, and even more preferably from 1 to 12. These are advantageously separated from one another so as to form separate elements.

Advantageously, the posterior portion (4) has (at least) 6 longitudinal elements (5) and the anterior portion (3) has (at least) 2 longitudinal elements (6). These are advantageously separated from one another so as to form separate elements.

These embodiments with regard to the number of longitudinal elements (5, 6) of the anterior (3) and posterior (4) portions can be applied to all the other preferred embodiments listed below.

This makes it possible to provide a device that takes up a certain amount of space, which makes it easier to handle.

In a particularly preferred manner, said at least one longitudinal element (6) of the anterior portion (3) and said at least two longitudinal elements (5) of the posterior portion (4) each have a hollow shape (7) arranged to allow blood flow, in the deployed position. This means that blood flow can take place through each of the aforementioned longitudinal elements.

Even more preferably, said at least one longitudinal element (6) has a hollow internal portion (13b) and a solid external portion (13a), the hollow internal portion (13b) defining a surface area that is larger than the surface area defined by the solid external portion (13a). The external portion (13a) takes the form of a segment extending around the circumference of said longitudinal element.

Advantageously, said at least two longitudinal elements (5) each have a hollow internal portion (12b) and a solid external portion (12a), the hollow internal portion (12b) defining a surface area that is larger than the surface area defined by the solid external portion (12a). The external portion (12a) is preferably in the form of a segment extending around the circumference of said longitudinal element.

Advantageously, the posterior portion (4) comprises a greater number of longitudinal elements (5) than the number of longitudinal elements (6) present in the anterior portion (3). This achieves a superior clinical effectiveness than that of known devices. This embodiment makes it possible to cover the entire posterior zone of the valve, which may have more or less numerous and more or less marked indentations, during delivery, which makes it possible to better manage all types of prolapses of the posterior leaflet of the valve.

In a particularly advantageous embodiment, in the deployed position, said distal ends (6b) of said at least 2 longitudinal elements (6) of the anterior portion (3) are in a position raised by a height of between 0.5 and 15 mm, preferably between 0.5 and 10 mm, more preferably between 3 and 10 mm relative to said central portion (2) which is located in a longitudinal axis (A1) relative to the length (L) of said body (1).

Thus, when the device is used to treat mitral regurgitation, the pressure applied to the anterior side of the mitral valve is such that the device can be effectively held in position over time.

Preferably, in the deployed position, said distal ends (5b) of said at least 2 longitudinal elements (5) of the posterior portion (4) being in a position raised by a height of between 0.5 and 15 mm, preferably between 0.5 and 10 mm, more preferably between 3 and 10 mm, relative to said central portion (2) which is located in a longitudinal axis (Al) relative to the length (L) of said body. Thus, when the device is used to treat mitral regurgitation, the pressure applied to the posterior side of the mitral valve is such that the device can be effectively held in position over time.

Advantageously, when the device according to the invention is in the deployed position on the mitral valve, all the ends (5b, 6b) of the respective longitudinal elements (5, 6) rest (in part) on the mitral annulus, while remaining free from one another. This means that there are no connecting means connecting said free ends of the longitudinal elements to each other when the device is in the deployed position on the mitral valve.

Advantageously, said at least one longitudinal element (6) of the anterior portion (3) and said at least two longitudinal elements (5) of the posterior portion (4) have a curved shape.

Advantageously, said central portion (2) is provided with an opening (11) arranged to receive a delivery means to bring the device above the mitral leaflets. This allows for easy positioning and improved clinical functionality

In this way, it is possible, for example, to insert a guide, such as a wire, designed to orient the device in the space, preferably at the level of the native mitral annulus.

Even more advantageously, said body (2) is molded in a single piece, preferably after laser or water jet cutting, in the form of a sheet of a material, advantageously with shape memory or superelastic, intended for medical use.

The shape can then be cut from a sheet of metal (e.g. nitinol) using a laser and then placed in a high-temperature thermoforming mold to then undergo very rapid quenching.

The body is advantageously made of a biocompatible material selected from the group comprising alloys, nitinol, titanium, stainless steel, polymer material, and mixtures thereof.

According to a particularly preferred embodiment, the thickness of the body (1) is between 0.01 and 1 mm, preferably between 0.1 and 0.5 mm, more preferably between 0.2 and 0.4 mm, even more preferably between 0.2 and 0.3, advantageously between 0.2 and 0.35 mm.

In an advantageous embodiment, said at least one longitudinal element (6) of the anterior portion (3) and said at least two longitudinal elements (5) of the posterior portion (4) have the shape of lobes, petals, or branches.

Preferably, said longitudinal elements (5) of the posterior portion (4) of said device, in the deployed position, extend so that the distal ends (5b) form an arc of a circle.

Advantageously, the distal end (6b) of said at least one longitudinal element (6) of the anterior portion (3) and each distal end (5b) of said at least two longitudinal elements (5) of the posterior portion (4) are provided with a fixing means or a receiving means (8, 9) arranged to receive an attachment means (10).

Preferably, the device according to the invention also comprises at least one attachment means (10) for fixing it to a native mitral annulus.

Thus, the method can advantageously comprise a step of positioning each fixing means or attachment means using fixing points which are located on the native mitral annulus.

During the step of delivering the device (which may be in the form of a grid) according to the invention, it is held in position and the behavior of the mitral valve and mitral leaflets are observed using imaging methods known to those skilled in the art. Thus, and if necessary, the device is repositioned until its functionality is confirmed. This is advantageously performed before fixing the device.

Then, as soon as the desired position is adopted, each fixing or attachment means is fixed to the mitral annulus so that it can function properly so as to treat mitral regurgitation.

In a particularly advantageous embodiment, the device is first held in the folded position within the first medical device in the form of a flexible tube (a first catheter). It then adopts the deployed position in which it can be placed on the mitral annulus. Optionally, if necessary, it is also possible after delivering and fixing the device, or even before delivery, to return to the folded position so the device can be easily removed from the body. The sequence of these folded, deployed, and folded positions ensures flexibility of use during delivery or even after delivery, allowing the device to be controlled, thereby limiting the risks associated with such an implantation procedure.

According to a preferred embodiment, the fixing means or the attachment means (10) of said at least one longitudinal element (6) of the anterior portion (3) are arranged to be placed above the anterior mitral leaflet and fixed to the anterior side of the mitral valve.

According to an even more preferred embodiment, the fixing means or the attachment means (10) of the at least two longitudinal elements (5) of the posterior portion (4) are arranged to be placed above the posterior mitral leaflet and fixed to the posterior side of the mitral valve.

According to an even more advantageous embodiment, said attachment means (10), arranged to be connected to said receiving means (8), has a distal attachment end (10a) arranged to be fixed to the native mitral annulus and a proximal end (10b), optionally provided with a connection means (10b) arranged to hold said attachment means (10) in said receiving means (8).

Preferably, said attachment means (10) has a first position arranged to allow it to be introduced into said receiving means (8) and a second position arranged to hold it in said receiving means (8), when it is fixed on the native mitral annulus in a fixed and stable manner over time.

According to a particular embodiment of the device according to the invention, in the folded position, it is arranged to be held in a medical device in the form of a flexible tube, preferably a catheter, which has an external diameter of between 3 and 15 mm, preferably between 4 and 12 mm, more preferably between 6 and 10 mm, and even more preferably between 6 and 8 mm.

Other embodiments of the method according to the invention will be detailed below.

According to a preferred embodiment of the device according to the invention, the latter is in the deployed position and has the following characteristics:
said posterior portion (4) being arranged to be positioned above the posterior mitral leaflet and arranged to be fixed on the posterior side of the mitral valve and comprising at least 2, preferably at least 3, longitudinal elements (5) each of which has a proximal end (5a) connected to said central portion (2) and a distal end (5b) relative to said central portion (2) so that said at least 3 longitudinal elements (5) each extend away from said central portion (2),
said anterior portion (3) being arranged to be placed above the anterior mitral leaflet and arranged to be fixed on the anterior side of the mitral valve and comprising at least 2 longitudinal elements (6) each of which has a proximal end (6a) connected to said central portion (2) and a distal end (6b) relative to said central portion (2) so that said at least 2 longitudinal elements (6) each extend away from said central portion (2) and also said at least 3 longitudinal elements (5), and in that
each of the distal ends (6b) of said at least 2 longitudinal elements (6) and/or each of said distal ends (5b) of said at least 3 longitudinal elements (5) being in a raised position relative to said central portion (2) which is located in a longitudinal axis (Al) relative to the length (L) of said body (1).

The device according to the invention can thus preferably adopt the following successive positions:
folded position,
deployed position,
return to folded position.

Other characteristics, details and advantages of the invention will be detailed in the description given below, by way of non-limiting example and with reference to the drawings and examples.

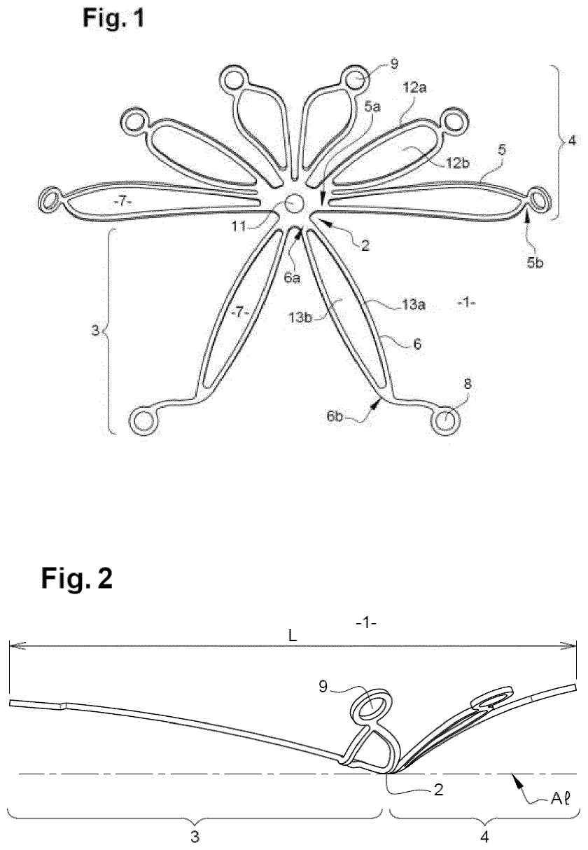

FIG. 1 is a top view of an embodiment of the device according to the present invention.

FIG. 2 is a side view of a preferred embodiment of the device according to the present invention.

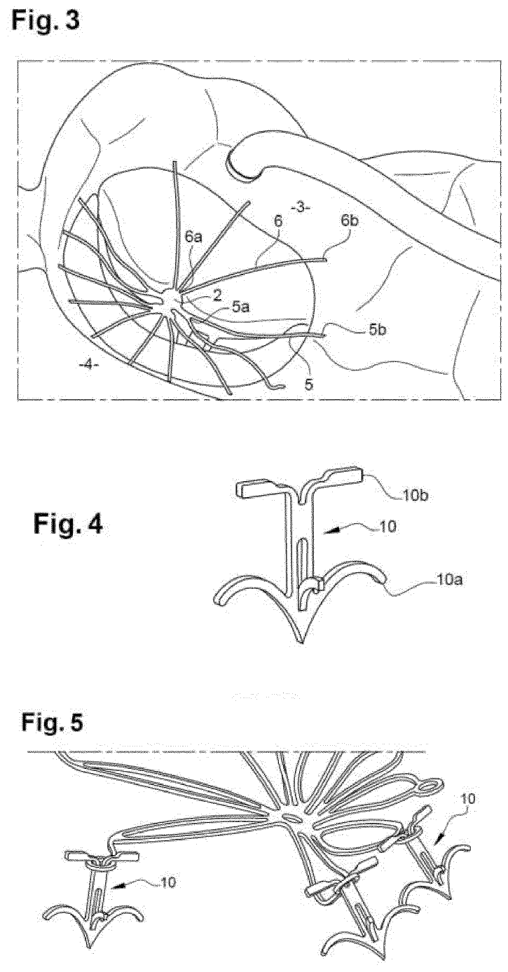

FIG. 3 is a top view of the device according to the invention when it is positioned at the native mitral annulus.

FIG. 4 is an illustration of an attachment means of the device according to the invention.

FIG. 5 is an illustration of an advantageous embodiment of the device according to the invention.

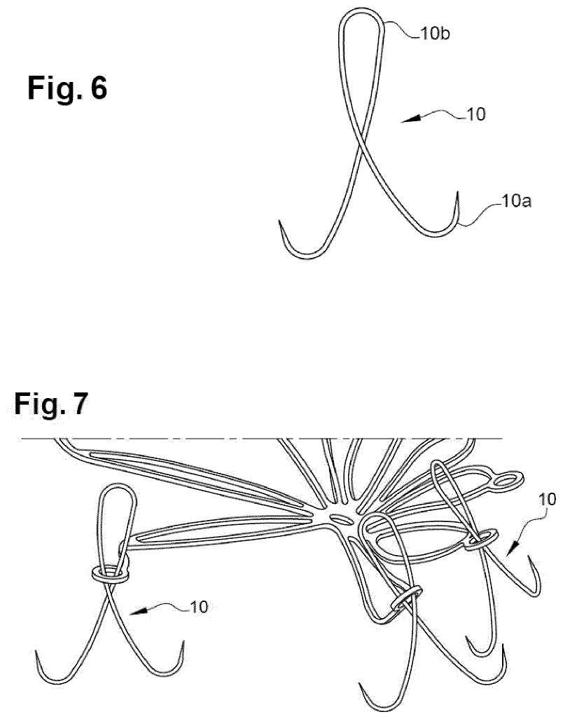

FIG. 6 shows an attachment means of the device according to the invention, and FIG. 7 illustrates this attachment means on the device of the invention.

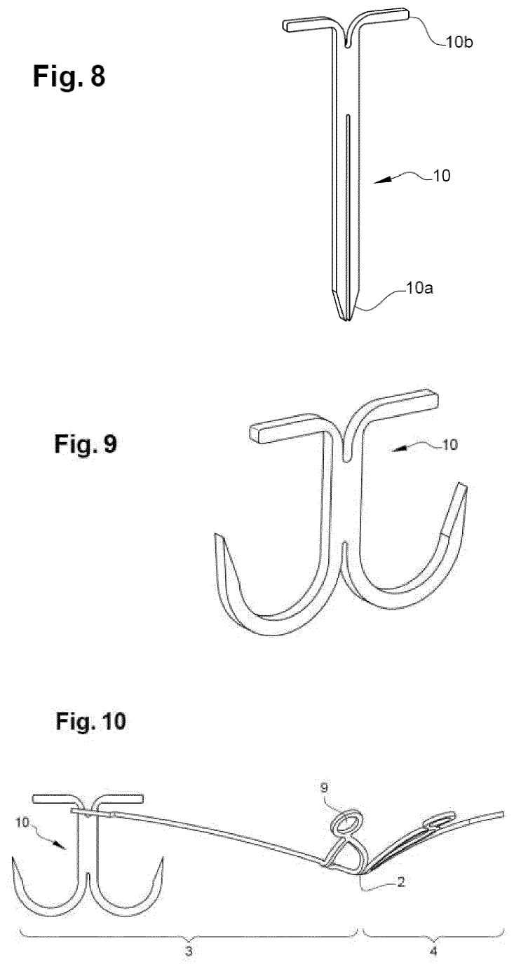

FIGS. 8 and 9 Show a Preferred Attachment Means.

FIG. 10 illustrates the attachment means of FIG. 9 when present on the device according to the invention.

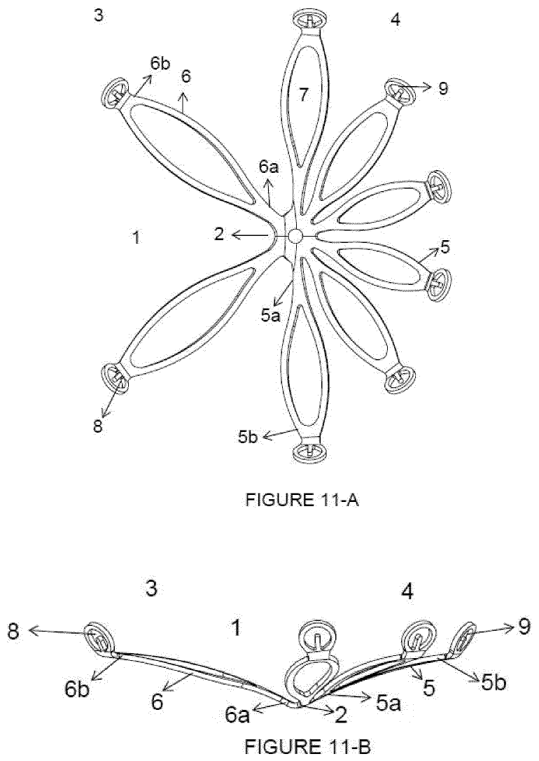

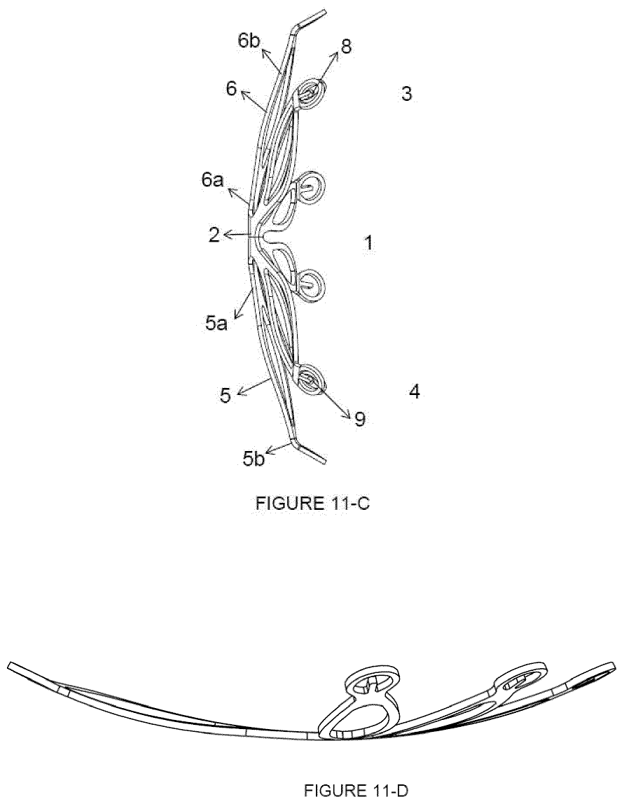

FIGS. 11-A, 11-B, 11-C, and 11-D illustrate an additional embodiment of the device according to the invention.

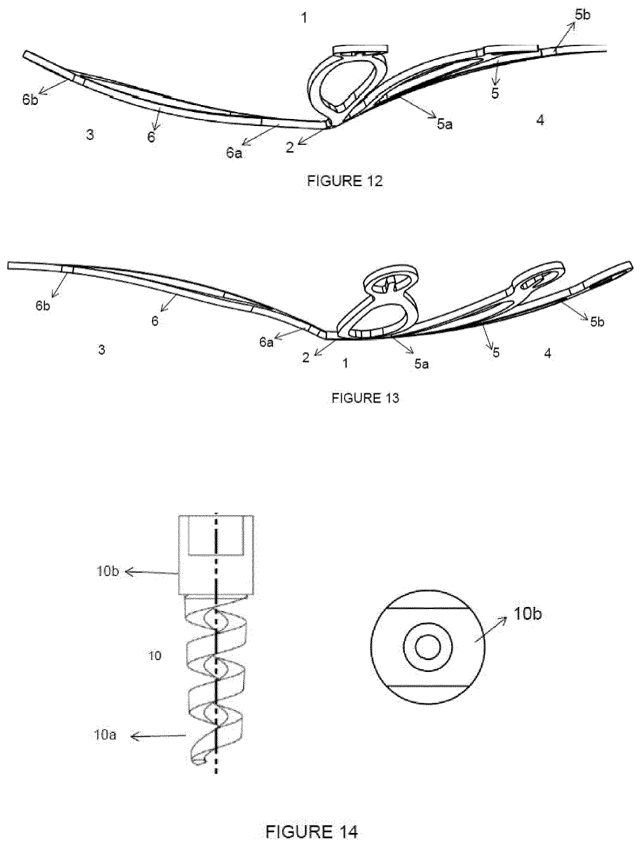

FIGS. 12 and 13 show two exemplary embodiments of the device according to the invention in two separate deployed positions.

FIG. 14 illustrates an attachment means of the device according to the invention.

FIG. 15 Is a Photograph of the Device in the Folded Position in a catheter.

In the figures, identical or similar elements have the same references. Other characteristics and advantages of the present invention will be detailed in the following non-limiting description, and with reference to the drawings and examples.

In the context of the present invention, the device provided may for example be positioned and fixed on an annulus which may have a D shape. This annulus may have a first diameter corresponding to a first position and a second diameter corresponding to a second position. Thus, when the annulus moves from the first position to the second position, the device according to the invention follows this movement while remaining securely and stably attached to said annulus. The first diameter is smaller than the second diameter of the aforementioned annulus. The annulus can thus move from the first position to the second position and vice versa. The device according to the invention is advantageous wherein it can follow these positions while remaining attached to the annulus.

Having the central portion (2) in a lower position relative to the distal ends, as indicated above, makes it possible to provide a device that exhibits a certain flexibility. Indeed, when the device according to the invention is positioned and fixed to an annulus (which may be D-shaped), the fixing of the distal ends (5b, 6b) to the periphery of the annulus allows the device to remain stably attached (over time) to said element. Also, if a fluid passes through the annulus to which the device according to the invention is attached, the structure of each longitudinal element (5, 6) allows the fluid to pass through, while ensuring fixation over time, which is particularly advantageous.

The annulus indicated above may advantageously be a native mitral annulus.

In the context of the present invention, the term ‘native’ refers to the organs of the heart. Thus, the ‘native’ mitral annulus refers to the annulus present in the left atrioventricular valve of the heart.

Thus, the at least two longitudinal elements (5) of the posterior portion (4) and/or the at least one longitudinal element (6) of the anterior portion (3) have a structure (hollow, see claims and figures) such that they allow a fluid to pass through, when the device is fixed to an annulus.

The central portion (2) has a first position corresponding to that described for the device in the deployed position. Thus, when a fluid passes through the grid (device according to the invention), the central portion (2) is arranged to adopt a second position corresponding to an inverted position (when the fluid passes through the grid with a certain force) wherein the central portion is raised relative to the distal ends (5b, 6b). It can thus slide along a vertical axis (perpendicular to the longitudinal axis A1) so as to reverse the configuration of the device according to the invention. The distal ends (5b, 6b) are then in a low position relative to the central portion (2), which is then raised.

Other configurations are also shown in FIGS. 12 and 13, where a different direction of curvature can be seen depending on the figure.

This flexibility is particularly preferred.

Preferably, the at least one longitudinal element (6) of the anterior portion (3) extends over a distance which is greater than that of the at least two longitudinal elements (5) of the posterior portion (4).

FIG. 1 illustrates a top view of an embodiment of the device according to the invention.

The device has a body (1) comprising a central portion (2) from which extends an anterior portion (3) and a posterior portion (4). The central portion (2) is provided with an opening (11) which is arranged to allow blood flow and also to facilitate handling of the device, for example by passing through a guide (a wire) which can be fixed to the device in order to move it in the space (left atrium of the heart).

Although the device has a folded position when housed in a medical device in the form of a flexible tube, such as a catheter, the illustrated embodiment relates to the deployed position which is arranged to treat mitral regurgitation.

In the deployed position, said posterior portion (4), arranged to be positioned above the posterior mitral leaflet and arranged to be fixed on the posterior side of the mitral valve, comprises six longitudinal elements (5). Each longitudinal element (5) has a proximal end (5a) connected to said central portion (2) and a distal end (5b), relative to said central portion (2), which is provided with a receiving means (9) arranged to receive an attachment means (not shown). Thus, the 6 longitudinal elements (5) each extend away from said central portion (2).

In this deployed position, said anterior portion (3), arranged to be positioned above the anterior mitral leaflet and arranged to be fixed to the anterior side of the mitral valve, comprises 2 longitudinal elements (6). Each longitudinal element (6) has a proximal end (6a) connected to said central portion (2) and a distal end (6b), relative to said central portion (2), which is provided with a receiving means (8) arranged to receive an attachment means (not shown). Thus, the two longitudinal elements (6) each extend away from said central portion (2) and also the six longitudinal elements (5).

As illustrated in FIG. 1, all the longitudinal elements (5, 6) are separated from one another in that they form separate elements and each have a hollow shape (7) that is arranged to allow blood flow. Thus, each longitudinal element (5, 6) comprises a hollow internal portion (12b, 13b) that defines a surface area greater than the surface area defined by the solid external portion (12a, 13a). The external portion (12a, 13a) is in the form of a segment extending around the circumference of a longitudinal element (5, 6). Thus, the longitudinal elements (5, 6) have the shape of hollow lobes.

The receiving means is not essential. Thus, the distal end of each longitudinal element (5, 6) may comprise a fixing means which is then arranged to fix the device to the native mitral annulus.

Although not visible in the figure, each of the distal ends (6b) of the two longitudinal elements (6) and each of said distal ends (5b) of the six longitudinal elements (5) are in a raised position relative to said central portion (2) which is located on a longitudinal axis (A1) relative to the length (L) of said body (1).

FIG. 2 illustrates the device described in FIG. 1 above. However, the illustration is a side view, which makes it possible to visualize how the device is arranged in the space

In this case, the distal ends (5b, 6b) of the longitudinal elements (5, 6) are in a raised position relative to said central portion (2) which is located in a longitudinal axis (A1) relative to the length (L) of said body (1).

The distal ends (5b, 6b) of the longitudinal elements (5, 6) are in a position raised by a height of between 3 and 10 mm, relative to said central portion (2) which is located in a longitudinal axis (A1) relative to the length (L) of said body (1).

Also, the longitudinal elements (5, 6) have a curved shape, as illustrated in FIG. 2.

FIG. 3 illustrates a top view when the device according to the invention is positioned and fixed on the native mitral annulus in the presence of the medical device, in this case a catheter.

FIG. 4 is a view of an attachment means (10) which has a V-shaped distal fixing end (10a) which is arranged to be fixed to the native mitral annulus, and a proximal end which is provided with a connection means (10b) which can be fitted in one of the receiving means of the device according to the invention. Thus, the connection means (10b) can be oriented in order to pass into a receiving means of the device, as illustrated in FIG. 5.

FIG. 5 represents a view in which the device according to the invention comprises 3 attachment means (10), shown in FIG. 4.

It should be noted that, preferably, the connection means (10b) and the distal attachment end (10a) extend in the same plane as that which passes

Through the portion of the attachment means (10) which is located between the 2 ends.

Furthermore, a protrusion extending away from the plane which passes through the connecting means (10b) and the distal attachment end (10a) makes it possible to ensure effective fixing.

FIG. 6 illustrates an attachment means (10) according to another variant of the invention. The attachment means (10) comprises a loop and two sharp ends that point towards said loop.

FIG. 7 is an embodiment in which three attachment means (10) are held in their respective receiving means of the device according to the invention.

FIG. 8 illustrates an additional variant of an attachment means (10) which has a distal attachment end (10a) and a proximal end provided with a connection means (10b).

It should be noted that, preferably, the connection means (10b) and the distal attachment end (10a) extend in the same plane as that which passes through the portion of the attachment means (10) which is located between the 2 ends. FIG. 8 illustrates the attachment means in a first position in which it can be inserted into the receiving means of the device according to the invention. Also, in this first position, the distal end of the attachment means extends away from the connection means (10b).

FIG. 9 illustrates the attachment means (10) of the variant shown in FIG. 8, in a second position, called the attachment position. In this second position, the distal attachment end extends towards the connection means. The distal attachment ends are sharp.

Thus, the attachment means of FIG. 8 is inserted into the receiving means (8) and then adopts the second position, as illustrated in FIG. 9.

FIG. 10 illustrates the device according to the invention when the attachment means (10) is connected to the receiving means (8).

FIG. 11-A includes all the elements described for FIG. 1. FIG. 11-B is another view of this device, and FIG. 11-C is a side view of the device according to the invention. All the elements described above are found in FIG. 11, with the exception of the attachment means. FIG. 11-D has a cup-shaped design.

FIGS. 12 and 13 illustrate two possible positions of the device according to the invention, with specific curvatures depending on clinical needs.

FIG. 14 illustrates an additional embodiment of an attachment means in the form of a screw, the proximal end (10b) of which is shown in a top view. This attachment means can be used to secure the device to an annulus, preferably a native mitral annulus, as explained in the scope of the present invention.

FIG. 15 is a photograph of an embodiment of the device according to the invention when it is in the folded position within a catheter.

In this embodiment, the central portion (2) is diametrically opposite the distal ends (5b, 6b) of the respective longitudinal elements (5, 6).

In the figures, identical or similar elements have the same references.

For the purposes of the present invention, any singular article, such as, for example, “a,” “an,” may be replaced by an article designating a plural, such as, for example, “at least 2,” “at least 3,” “several,” etc.

The word “include,” “contains,” or any equivalent or derivative term may be replaced by “consisting of” in order to define a list or exclusive selection of possibilities so as not to encompass other elements not mentioned in the expression used.

For the purposes of the present invention, any range of values listed above includes the limit values indicated. Thus, when “between 1 and 10” is indicated, for example, the values 1 and 10 are also included.

It is understood that the present invention is in no way limited to the embodiments described above and that many modifications may be made thereto without departing from the scope of the appended claims.

1. A device arranged to allow transcatheter implantation, said device comprising a body (1) having a central portion (2) from which extends an anterior portion (3) and a posterior portion (4), said device having a folded position arranged to be held in a medical device in the form of a flexible tube, optionally a catheter, and a deployed position arranged to treat mitral regurgitation, and, wherein, in the deployed position:
said posterior portion (4) being arranged to be placed above the posterior mitral leaflet and arranged to be fixed on the posterior side of the mitral valve and comprising at least 2 longitudinal elements (5), each of which has a proximal end (5a) connected to said central portion (2) and a distal end (5b) relative to said central portion (2) so that said at least 2 longitudinal elements (5) each extend away from said central portion (2),
said anterior portion (3) being arranged to be placed above the anterior mitral leaflet and arranged to be fixed on the anterior side of the mitral valve and comprising at least 1 longitudinal element (6) which has a proximal end (6a) connected to said central portion (2) and a distal end (6b) relative to said central portion (2) so that said at least 1 longitudinal element (6) extends away from said central portion (2) and also said at least 2 longitudinal elements (5), and wherein
said distal end (6b) of said at least 1 longitudinal element (6) and/or each of said distal ends (5b) of said at least 2 longitudinal elements (5) being in a raised position relative to said central portion (2) which is located in a longitudinal axis (A1) relative to the length (L) of said body (1).

2. The device according to claim 1, wherein said at least 2 longitudinal elements (5) are separated from each other so as to form separate elements.

3. The device according to claim 1, wherein said at least 1 longitudinal element (6) and said at least 2 longitudinal elements (5) each have a hollow shape (7) arranged to allow blood flow, in the deployed position.

4. The device according to claim 1, wherein said posterior portion (4) comprises a greater number of longitudinal elements (5) than the number of longitudinal elements (6) present in said anterior portion (3).

5. The device according to claim 1, wherein, in the deployed position, said distal end (6b) of said at least 1 longitudinal element (6) being in a position raised by a height of between 0.5 and 15 mm, optionally between 0.5 and 10 mm, or optionally between 3 and 10 mm, relative to said central portion (2) which is located in a longitudinal axis (A1) relative to the length (L) of said body (1).

6. The device according to claim 1, wherein, in the deployed position, said distal ends (5b) of said at least 2 longitudinal elements (5) are in a position raised by a height of between 0.5 and 15 mm, optionally between 0.5 and 10 mm, or optionally between 3 and 10 mm, relative to said central portion (2) which is located in a longitudinal axis (A1) relative to the length (L) of said body.

7. The device according to claim 1, wherein, when said device is in the deployed position on the mitral valve, all the ends (5b, 6b) of the respective longitudinal elements (5, 6) rest on the mitral valve, while remaining free from each other.

8. The device according to claim 1, wherein said at least one longitudinal element (6) and said at least two longitudinal elements (5) have a curved shape.

9. The device according to claim 1, wherein said central portion (2) is provided with an opening (11) arranged to receive a delivery means for bringing said device above the mitral leaflets and for allowing blood flow.

10. The device according to claim 1, wherein said body (2) is molded in one piece, optionally after laser or water jet cutting, in the form of a sheet of a material, optionally with shape memory or superelastic, intended for medical use.

11. The device according to claim 1, wherein the thickness of said body (1) is between 0.01 and 1 mm, optionally between 0.1 and 0.5 mm, optionally between 0.2 and 0.4 mm, optionally between 0.2 and 0.3 mm, or optionally between 0.2 and 0.35 mm.

12. The device according to claim 1, wherein said at least one longitudinal element (6) and said at least two longitudinal elements (5) have the shape of a lobe, petal or branch.

13. The device according to claim 1, wherein each distal end (6b) of said at least one longitudinal element (6) and said at least two longitudinal elements (5) is provided with a fixing means or a receiving means (8, 9) arranged to receive an attachment means (10).

14. The device according to claim 13, wherein said attachment means (10) arranged to be connected to said receiving means has a distal attachment end arranged to be fixed to the native mitral annulus and a proximal end optionally provided with a connection means (10b) arranged to hold said attachment means (10) in said receiving means (8).

15. The device according to claim 1, wherein, in the folded position, said device is arranged to be held in a medical device in the form of a flexible tube, optionally a catheter, which has an external diameter of between 3 and 15 mm, optionally between 4 and 12 mm, optionally between 6 and 10 mm, or optionally between 6 and 8 mm.

16. A kit comprising the device according to claim 1 and a medical device in the form of a flexible tube, optionally a catheter, having an external diameter of between 3 and 15 mm, optionally between 4 and 12 mm, optionally between 6 and 10 mm, or optionally between 6 and 8 mm, wherein said device is held in the folded position in said medical device.

17. A method for transcatheter implantation of a medical device, the method comprising the following steps:
providing a the device which comprises a body (1) having a central portion (2) from which an anterior portion (3) and a posterior portion (4) extend, said body (1) being in a folded position in which said body (1) is held in a medical device in the form of a flexible tube, optionally a catheter,
bringing the device in the folded position into the left atrium of the heart by the venous, transfemoral or transseptal route,
deployment of the device so that it adopts a deployed position and orientation thereof above the mitral valve with
said posterior portion (4) of said body (1) being placed above the posterior mitral leaflet and fixed on the posterior side of the mitral valve (or native mitral annulus), said posterior portion (4) comprising at least 2 longitudinal elements (5) each of which has a proximal end (5a) connected to said central portion (2) and a distal end (5b) relative to said central portion (2) so that said at least 2 longitudinal elements (5) each extend away from said central portion (2), and with
said anterior portion (3) which is placed above the anterior mitral leaflet and arranged to be fixed on the anterior side of the mitral valve (or idem), said anterior portion (3) comprising at least 1 longitudinal element (6) which has a proximal end (6a) connected to said central portion (2) and a distal end (6b) relative to said central portion (2) so that said at least 1 longitudinal element (6) extends away from said central portion (2) and also said at least 2 longitudinal elements (5), and
stable delivery of the device above the mitral leaflets and removal of said medical device in the form of a flexible tube.

18. The device according to claim 1, wherein the medical device in the form of a flexible tube is a catheter.

19. The kit according to claim 16, wherein said medical device in the form of a flexible tube is a catheter.

20. The method according to claim 17, wherein said medical device in the form of a flexible tube is a catheter.
